(19) United States
(12) Patent Application Publication
(10) Pub. No.: US 2009/0236253 A1
(43) Pub. Date: Sep. 24, 2009

(54) STERILIZED PACKAGE, METHOD FOR ITS PRODUCTION, AND ITS USE IN MEDICINE

(76) Inventors: Christof Merckle, Mannheim (DE); Volker Friedrich, Neuhausen (DE)

Correspondence Address: IP GROUP OF DLA PIPER LLP (US), ONE LIBERTY PLACE, 1650 MARKET ST, SUITE 4900, PHILADELPHIA, PA, 19103, US

(21) Appl. No.: 12/303,275

(22) PCT Filed: Jun. 4, 2007
(86) PCT No.: PCT/EP2007/004942

§ 371 Date: Dec. 3, 2008

(30) Foreign Application Priority Data
Jun. 6, 2006 (DE) 10 2006 027 304.4

(51) Int. Cl. A61B 19/02

(52) U.S. Cl. 206/439; 53/403

(57) ABSTRACT

A sterilized package includes an inner gas-permeable and germ-proof package which contains at least one sterile article and an outer gas-tight package which surrounds the inner package wherein the package includes, between the inner package and the outer package, a sterile and dry packaging volume.

### RELATED APPLICATIONS

This is a §371 of International Application No. PCT/EP2007/004942, with an international filing date of Jun. 4, 2007 (WO 2007/140970 A1, published Dec. 13, 2007), which is based on German Patent Application No. 10 2006 027 304.4, filed Jun. 6, 2006.

### TECHNICAL FIELD

This disclosure relates to a sterilized package, comprising an inner gas-permeable and germ-proof packaging means, which contains at least one sterile article, and comprising an outer gas-tight packaging means, to a method for producing the sterilized package, and to its use for dry and sterile storage of the article, and also to its use for sterile removal of the inner packaging means from the package.

### BACKGROUND

The packaging and sterilization of products is of considerable importance particularly in the field of medicine. The products are usually sterilized before or during the packaging procedure. The choice of sterilization method depends essentially on the material from which the products to be packaged are made. A sterilization method that is frequently used is gamma irradiation. However, some materials, for example polymers based on glycolide and/or p-dioxanone, decompose if exposed to gamma rays for prolonged periods. In these cases, gentler sterilizing conditions have to be employed. For example, a suitable treatment for this purpose is to use a sterilization gas, in particular ethylene oxide.

The products that are to be packaged and sterilized are often transferred into a suitable package and then sterilized, in which case the package is closed only after sterilization. A disadvantage of this, however, is that the sterilization gas is removed after sterilization has ended, at least in those cases where residues of the sterilization gas may damage the sterilized products before the package is closed. During the removal of the sterilization gas, additional safety measures are often needed to maintain the desired sterility.

For this reason, products are increasingly being enclosed in suitable packages and then subjected to a sterilization process. For this purpose, packages are used that are made at least partially of a gas-permeable material that is at the same time impermeable to pathogens. Materials of this kind are commercially available under the trade name Tyvek®, for example.

A method for packaging and sterilization of articles is known from US 2005/0268573 A1, in which the articles are first enclosed in a package. The package is at least partially permeable to gases, but not to microbial pathogens. The package, including the article located therein, is then treated with a sterilization gas. After sterilization, the package is transferred into a second impermeable package, for example, and likewise enclosed therein. A problem, however, is the necessity for the second packaging step, which also has to be performed under strictly controlled conditions to ensure that no traces of sterilization gases or of moisture are entrained in the second packaging step. A sterilized dual package that can be produced in one sterilization step is described in U.S. Pat. No. 4,482,053.

In view of the ever increasing demands placed on the sterility and the quality of packaged products, in particular in the medical sector, there is a continuing need for sterilized packaging systems that meet these demands and that contribute in particular to improved medical safety standards.

It could therefore be helpful to provide a sterilized package and a corresponding sterilization method that each meet the above-mentioned demands and that in particular avoid the disadvantages of the prior art.

### SUMMARY

We provide a sterilized package including an inner gas-permeable and germ-proof package which contains at least one sterile article; an outer gas-tight package which surrounds the inner package; and a sterile and dry packaging space between the inner package and the outer package.

We also provide a method for producing the sterilized package including surrounding the inner package with an outer and as yet unclosed package, closing a germ-proof and gas-permeable closure of the inner package, sterilizing an interior portion of the inner package containing the at least one sterile article and an interior portion of the outer package by introducing a sterilization gas, and closing the outer package.

### BRIEF DESCRIPTION OF THE DRAWING

Further details and features will become clear from the following description of representative structures and from the Drawing and the Example. In these structures, individual features can each be realized on their own or in combination with other features. By express reference, the figure is herewith made part of the content of this description.

The Drawing is a schematic side view of one of our packages, taken partially in section for ease of understanding.

### DETAILED DESCRIPTION

We provide a sterilized package comprising an inner gas-permeable and germ-proof packaging means, which contains at least one sterile article, and comprising an outer gas-tight packaging means which surrounds the inner packaging means, wherein the package comprises, between the inner packaging means and the outer packaging means, a sterile and dry packaging volume.

We provide sterilized packages that have an inner packaging means and an outer packaging means, wherein the inner packaging means has a sterile and dry outside. The article is kept dry and sterile in the inner packaging means. The dry and sterile interior of the inner packaging means is supplemented by what is preferably also a dry and sterile interior of the outer vapor-impermeable packaging means. This has the particular advantage of contributing to increased storage stability of the article. The packages-also allow the inner packaging means to be removed in a sterile manner with the sterile article from the package. There is a great need to be able to do this, particularly in the medical sector. Thus, the packages contribute to improved sterilization standards and additionally provide improved safety against possible contamination of medical treatment rooms, in particular of operating theaters, with pathogens.

“Germs” as used herein are understood to mean all microbial pathogens, in particular bacteria and viruses.

In one aspect, at least one gas-permeable and germ-proof sterilization opening is located inside a gas-tight wall of the outer packaging means, in particular inside the outer packaging means. The sterilization opening is preferably in the form of a wall part. Two gas-permeable and germ-proof sterilization openings are expediently located inside the gas-tight wall of the outer packaging means.

In a further aspect, a sterilization opening of the package is formed by a gas-permeable and germ-proof wall, in particular a wall part, of the outer packaging means, and the sterilization opening itself is closed in a gas-tight manner. In another aspect, a sterilization opening of the package is formed by a gas-permeable and germ-proof wall, in particular a wall part, of the inner packaging means.

It is particularly preferable for both the inner and also the outer packaging means to have a respective gas-permeable and germ-proof sterilization opening. The sterilization openings can in particular be arranged over each other. The sterilization openings can have substantially identical dimensions, for example. It is also possible for the sterilization openings to be connected to each other, in particular to be adhesively bonded or sealed onto each other. Thus, in particular, the sterilization opening of the outer packaging means can be applied onto the sterilization opening of the inner packaging means. However, the sterilization openings are preferably formed spatially separate from each other.

The gas-tight wall of the package is preferably a film, in particular a cover film. The gas-tight wall can comprise metals or composite materials. Aluminum in particular can be used as the metal. As for composite materials, it is possible in principle to use all materials familiar to those skilled in the art. Composite materials that contain aluminum as a component of the composite are particularly suitable by virtue of the vapor impermeability of aluminum. The composite materials can in particular have a layered structure. The composite materials can comprise sealing layers or adhesive layers, for example. Advantageously, the composite materials comprise laminates, in particular based on plastics. The gas-tight wall of the package is preferably formed from composite materials based on metals, paper, plastics and/or lacquers. The composite materials can for example be formed from the following components: polyethylene, polypropylene, polyethylene terephthalate, polyamide, polyvinyl chloride, lacquer, paper and/or aluminum. Particular preference is given to the following compositions:
polyethylene/aluminum/polyethylene terephthalate,
polyvinyl chloride/aluminum/polyamide,
polypropylene/aluminum/lacquer,
polyethylene terephthalate/aluminum/polypropylene, and
lacquer/aluminum/polyethylene terephthalate/paper.

In one aspect, the sterilization opening may be a gas-permeable film. The gas-permeable film can in particular be a cover film or masking film. The package preferably has two gas-permeable films, in particular a gas-permeable masking film and a gas-permeable cover film. The gas-permeable masking film is in particular a sterilization opening of the inner packaging means, and the gas-permeable cover film is preferably a sterilization opening of the outer packaging means.

In a further aspect, the package may have a total of three films, of which two films in particular are gas-permeable. Provision may also be made for two films to be configured as cover films. The films are preferably a gas-permeable masking film, a gas-permeable cover film and a gas-tight cover film. For further features and details of the gas-permeable films, reference is made to the preceding paragraph. The gas-permeable cover film and the gas-tight cover film are preferably more or less identical in terms of their dimensions. The gas-tight cover film can in particular be applied onto the gas-permeable cover film. Application techniques that can be used are, in particular, adhesive bonding and sealing techniques. For example, sealing seams, in particular weld seams, of the gas-tight cover film can extend on the gas-permeable cover film or extend parallel to the gas-permeable cover film. The package can also have a peel area, in particular for easier handling. The films, in particular the cover films, can expediently be welded together in the peel area of the package.

In another aspect, the sterilization opening of the package may be made from a non-woven material, in particular from a nonwoven polymer material. The nonwoven material is preferably made from polyolefin, in particular from polyethylene or polypropylene, preferably polyethylene. The nonwoven material can also be a high pressure polymer, in particular high pressure polyethylene. For example, the nonwoven material can be one of the products commercially available under the trade names Tyvek® or Typar®.

The inner and/or the outer packaging means, preferably the inner and the outer packaging means, are expediently free of sterilization gases. In this way, it is possible to avoid undesired damage to the sterile article and, consequently, avoid a possible loss of quality of the article.

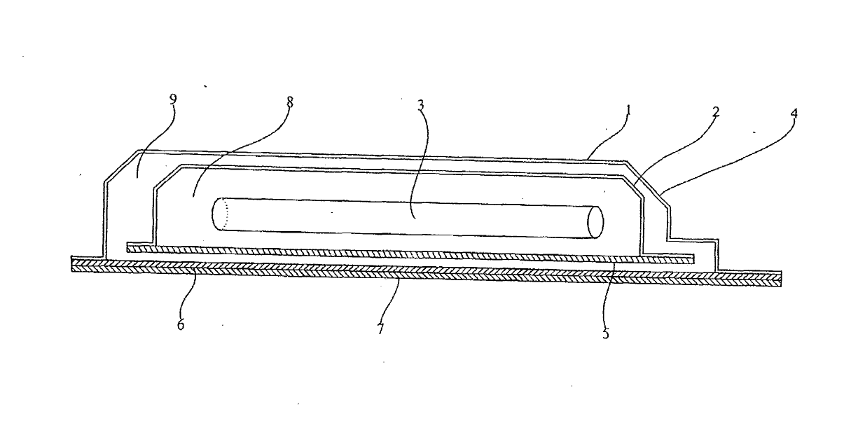

In another aspect, the inner and/or the outer packaging means, preferably the inner and the outer packaging means, are made at least partly from dimensionally stable materials. The dimensionally stable materials are preferably plastic materials, in particular polyethylene terephthalate (PET) and/or polycarbonate. The dimensionally stable materials are preferably what are called “blister materials.” The packaging means can be present in different three-dimensional shapes and in particular have the shape of a housing. An example of one such housing shape is shown in FIG. 1. Provision can also be made for the inner and the outer packaging means of the package to each have different three-dimensional shapes. For example, the inner packaging means can be present in the form of a tube, and the outer packaging means in particular in the form of a blister. The tube can be designed such that it can be closed, for example by plugs. Moreover, the plugs can each have at least one gas-permeable and germ-proof sterilization opening.

The article in the package is preferably an article that is sensitive to moisture, in particular sensitive to hydrolysis. The article can in particular be a medical article, for example an implant, The article can be in the form of screws, clips, suture materials or vascular prostheses. The article is preferably a vascular prosthesis, which in particular can have a resorbable polymer coating.

In one aspect, the sterile assurance level (SAL) of the interiors of the inner and outer packaging means is in each case <10⁻³, preferably ≦10⁻⁶.

We further provide a method for producing a sterilized package comprising the following steps:
surrounding an inner packaging means with an outer and as yet unclosed packaging means,
germ-proof and gas-permeable closure of the inner packaging means,
sterilization of the interior of the inner packaging means, of at least one sterile (and in particular moisture-sensitive) article contained therein, and of the interior of the outer packaging means by introduction of a sterilization gas,
closure of the outer packaging means to form the package.

In one aspect, the article is transferred into the inner packaging means and, after transfer of the article, the inner packaging means is closed in a gas-permeable and germ-proof manner before the inner packaging means is surrounded by the outer packaging means. The inner packaging means is preferably closed by a gas-permeable and germ-proof material, in particular in the form of a gas-permeable and germ-proof film, preferably in the form of a masking film.

In a further aspect, after it has surrounded the inner packaging means, and before the sterilization, the outer packaging means is closed by a gas-permeable and germ-proof material, in particular in the form of a gas-permeable and germ-proof film, preferably in the form of a cover film or masking film. The outer packaging means is expediently closed off by a gas-permeable and germ-proof cover film. During the sterilization procedure, the sterilization gas is thus able to infiltrate through all of the film surface, in particular through all of the film surfaces of the inner and the outer packaging means and penetrate into the package and ensure sterilization of the interiors of the package and the article.

In a particularly preferred aspect, after the sterilization, the outer packaging means is closed by a gas-tight material, in particular in the form of a gas-tight film, preferably in the form of a gas-tight cover film. In this way, the sterility and dryness of the package, in particular of the interior of the inner packaging means and the interior of the outer packaging means, is maintained.

Ethylene oxide may be used as sterilization gas for sterilizing the package. After the package has been sterilized, the sterilization gas is preferably removed. The removal of the sterilization gas can be carried out for a period of between about 10 minutes and up to 7 days. To accelerate the removal of the gas, the sterilized package can be subjected to several vacuum cycles. The vacuum cycles can be carried out at regular or irregular intervals. The sterilization gas is preferably removed with simultaneous drying of the sterilized package. The sterilized package is expediently dried at a temperature of between about 25° C. and about 200° C., preferably of between about 30° C. and about 150° C., in particular of between about 30° C. and about 60° C. The drying can take place at normal pressure or at an underpressure. In the case of drying under vacuum, the normal pressure can be re-established by delivery of an inert gas, in particular by delivery of dried air or a dried protective gas. Examples of protective gases that can be used are argon and nitrogen.

We further provide for the use of the package for dry and sterile storage of the article and for sterile removal of the inner packaging means from the package, in particular for sterile removal of the article from the inner packaging means.

The Drawing shows a package (1) composed of an inner blister (2), which is designed as an inner packaging means, and an outer blister (4), which is designed as an outer packaging means. The inner blister (2) and the outer blister (4) each have a housing shape, the housing shape of the outer blister (4) being designed such that it can easily receive the housing shape of the inner blister (2). A vascular prosthesis (3) is stored as an article inside the inner blister (2). The inner blister (2) has a gas-permeable and germ-proof sterilization opening in the form of a masking film (5) made of Tyvek®. The outer blister (4) has a cover film (6) made of Tyvek® and designed as a gas-permeable and germ-proof sterilization opening. The cover film (6) is closed in a gas-tight manner by a cover film (7), which is designed as a gas-tight wall of the outer blister (4) and is made of a composite material based on aluminum, paper and polyethylene terephthalate (PET). The packaging volumes (8) and (9) are each sterile and free of residual moisture.

### Example 1

### Packaging of a Vascular Prosthesis Having a Synthetically Resorbable Polymer Coating of Poly-D,L-Lactide-Co-Glycolide (50:50)

A vascular prosthesis having a synthetically resorbable polymer coating is placed in an inner blister made of polyethylene terephthalate (PET). The inner blister is then sealed by a masking film made of Tyvek®. The sealed inner blister is transferred into an outer blister. After this transfer, the outer blister is sealed with a cover film made of Tyvek®. The package is then subjected to sterilization with ethylene oxide. After the sterilization procedure has been completed, the package is dried at a temperature of about 50° C. for a period of about 6 hours by gas exchange via all of the Tyvek® film, both of the inner blister and of the outer blister. Drying is accelerated by regular vacuum cycles, which are each maintained for about 15 minutes. After drying is complete, the outer blister is sealed in a gas-tight manner by a cover film made of a layered composite material based on aluminum, paper and polyethylene. The outer blister is sealed in a gas-tight manner by means of the layered gas-tight cover film being welded onto the Tyvek® cover film of the outer blister.

1-21. (canceled)

22. A sterilized package comprising:
an inner gas-permeable and germ-proof package which contains at least one sterile article;
an outer gas-tight package which surrounds the inner package; and
sterile and dry packaging space between the inner package and the outer package.

23. The package of claim 22, further comprising at least one gas-permeable and germ-proof sterilization opening located inside a gas-tight wall of the outer package.

24. The package of claim 22, further comprising a sterilization opening formed by a gas-permeable and germ-proof wall of the outer package, wherein the sterilization opening is closed in a gas-tight manner.

25. The package of claim 22, further comprising a sterilization opening in the inner package formed by a gas-permeable and germ-proof wall.

26. The package of claim 25, wherein the gas-tight wall is a cover film.

27. The package of claim 25, wherein the gas-tight wall comprises metals or composite materials.

28. The package of claim 24 or 25, wherein the sterilization opening is a gas-permeable cover film or masking film.

29. The package of claim 24 or 25, wherein the sterilization opening is a nonwoven polymer material.

30. The package of claim 29, wherein the nonwoven material is high pressure polyethylene.

31. The package of claim 22, wherein the inner package and/or the outer package are free of sterilization gases.

32. The package of claim 22, wherein the inner package and/or the outer package are made at least partly from dimensionally stable blister materials.

33. The package of claim 22, wherein the article is sensitive to moisture.

34. The package of claim 22, wherein the article is a vascular prosthesis.

35. A method for producing the sterilized package of claim 22, comprising:
surrounding the inner package with an outer and as yet unclosed package;
closing a germ-proof and gas-permeable closure of the inner package;
sterilizing an interior portion of the inner package containing the at least one sterile article and an interior portion of the outer package by introducing a sterilization gas; and
closing the outer package.

36. The method of claim 35, wherein the article is transferred into the inner package, and the inner package is closed in a gas-permeable and germ-proof manner before the inner package is surrounded by the outer package.

37. The method of claim 35, wherein the inner package is closed by a gas-permeable and germ-proof masking film.

38. The method of claim 35, wherein, after surrounding the inner package and before sterilizing, the outer package is closed by a gas-permeable and germ-proof cover film.

39. The method of claim 35, wherein, after sterilization, the outer package is closed by a gas-tight cover film.

40. The method of claim 35, wherein ethylene oxide sterilizes the interior portions.

41. The method of claim 25, wherein the sterilization gas is removed, optionally with simultaneous drying, at a temperature of between about 25° C. and about 200° C.
